(19) United States
(12) Patent Application Publication
(10) Pub. No.: US 2013/0066152 A1
(43) Pub. Date: Mar. 14, 2013

(54) Tracheal Intubation Device

(71) Applicant: Tien-Sheng Chen, Taipei City (TW)

(72) Inventor: Tien-Sheng Chen, Taipei City (TW)

(21) Appl. No.: 13/366,413

(22) Filed: Feb. 6, 2012

(30) Foreign Application Priority Data
Sep. 9, 2011 (TW) 100217028

(51) Int. Cl. A61B 1/267

(52) U.S. Cl. 600/188

(57) ABSTRACT

A tracheal intubation device includes a handle portion, a display unit, a bar-shaped camera, and a guiding portion. The display unit is electrically connected to the bar-shaped camera. The bar-shaped camera is used for capturing images and includes a camera rear end and a camera front end. The camera rear end is electrically connected to the display unit for displaying images captured by the bar-shaped camera. The camera front end has a bended portion. The guiding portion includes a front end and a rear end, wherein the front end is detachably connected to the handle portion. The guiding portion is used for accommodating the bar-shaped camera and the endotracheal tube to allow the bar-shaped camera and the endotracheal tube to move within the guiding portion.

### BACKGROUND OF THE INVENTION

1. Field of the Invention

The present invention relates to a medical device; more specifically, the present invention relates to a tracheal intubation device.

2. Description of the Related Art

For apnea patients, tracheal intubation is an essential operation for sustaining respiration to maintain the vital signs of a patient. Medical personnel have to place the endotracheal tube into the trachea of a patient to ensure that the oxygen supply is provided in a short period of time. Therefore, it is an important task for medical personnel to place the endotracheal tube efficiently. In practice, tracheal intubation devices equipped with a camera are often used by medical personnel to confirm the location of the trachea of a patient and then to place the endotracheal tube into the trachea of the patient. Tracheal intubation devices disclosed in U.S. Pat. No. 4,306,547 and US patent application number 2007/0106121 are the most widely used tracheal intubation devices, and the image capturing device of each tracheal intubation device is installed on the lateral side of the endotracheal tube slot. When the glottis of the trachea of the patient is observed via the image capturing device, medical personnel push the endotracheal tube installed on the lateral side of the image capturing device into the trachea of the patient.

Although the tracheal intubation device disclosed in US patent application number 2007/0106121 does improve the tracheal intubation, some drawbacks during operation of the device need to be improved. Sometimes, due to the physical structure of a patient, the visual field of this kind of tracheal intubation device is blocked by the parallax caused by the camera installed on the lateral side of the endotracheal tube slot or the endotracheal tube. Consequently, since the glottis of the trachea cannot be observed, the endotracheal tube cannot be precisely placed into the trachea of a patient by medical personnel. The Acta Anaethesiologica Scandinavica published in 2010 (2010; 54(9):1050-61) also indicates that the widely used tracheal intubation devices may occasionally fail in precise placement.

In view of the fact that efficient intubation is critical for the survival of a patient, there is a need to provide an intubation device by which an intubation can be performed easily and precisely.

### SUMMARY OF THE INVENTION

It is an object of the present invention to provide a tracheal intubation device by which an intubation operation can be carried out easily and precisely.

To achieve the abovementioned object, the tracheal intubation device of the present invention includes a handle portion, a display unit, a bar-shaped camera, and a guiding portion. The display unit is electrically connected to the bar-shaped camera. The bar-shaped camera is used for capturing images and includes a camera rear end and a camera front end. The camera rear end is electrically connected to the display unit for displaying images captured by the bar-shaped camera. The camera front end has a bended portion. The guiding portion includes a front end and a rear end, wherein the rear end is detachably connected to the handle portion. The guiding portion is used for accommodating the bar-shaped camera and the endotracheal tube and allows the bar-shaped camera and the endotracheal tube to move within the guiding portion.

Because the structure of the present invention is novel and useful in the industry due to its improvement, the present invention is entitled to apply for a utility patent.

### BRIEF DESCRIPTION OF THE DRAWINGS

FIG. 1 is a schematic drawing illustrating the combination of the tracheal intubation device of the present invention and the endotracheal tube.

FIG. 2 is a schematic drawing of one embodiment of the camera front end of the present invention before being pushed by the endotracheal tube.

FIG. 3 is a schematic drawing of one embodiment of the camera front end of the present invention after being pushed by the endotracheal tube.

FIG. 4 is a schematic drawing illustrating the usage state of one embodiment of the tracheal intubation device of the present invention.

FIG. 5 is a schematic drawing of another embodiment of the camera front end before being pushed by the endotracheal tube.

FIG. 6 is a schematic drawing of another embodiment of the camera front end after being pushed by the endotracheal tube.

### DETAILED DESCRIPTION OF THE PREFERRED EMBODIMENT

The advantages and innovative features of the invention will become more apparent from the following detailed descriptions when taken together with the accompanying drawings.

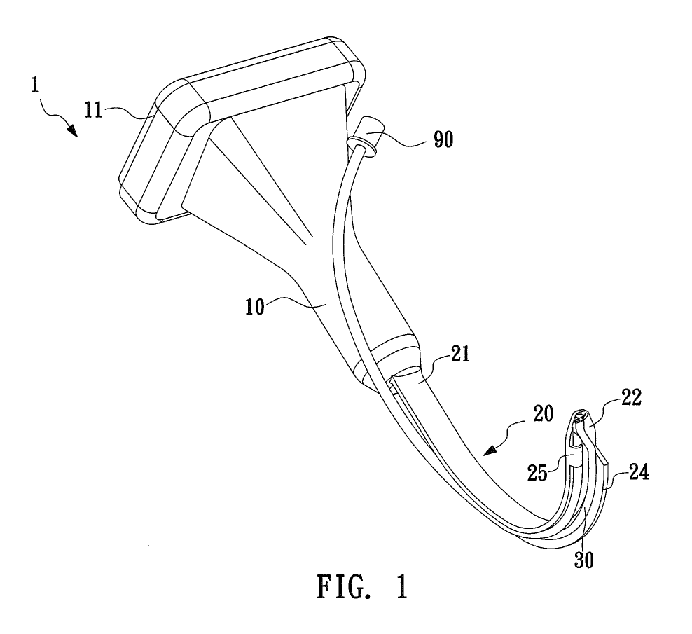

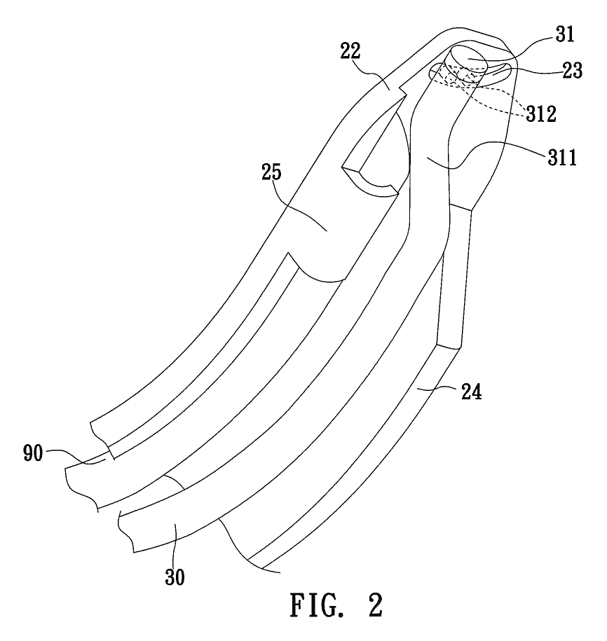

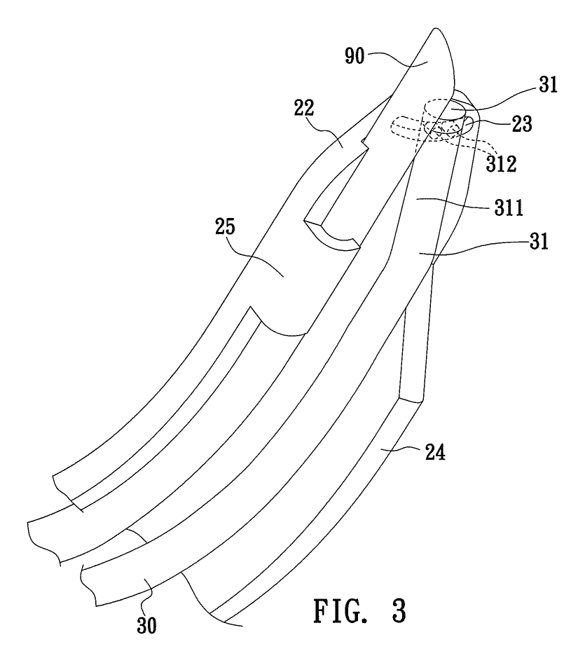

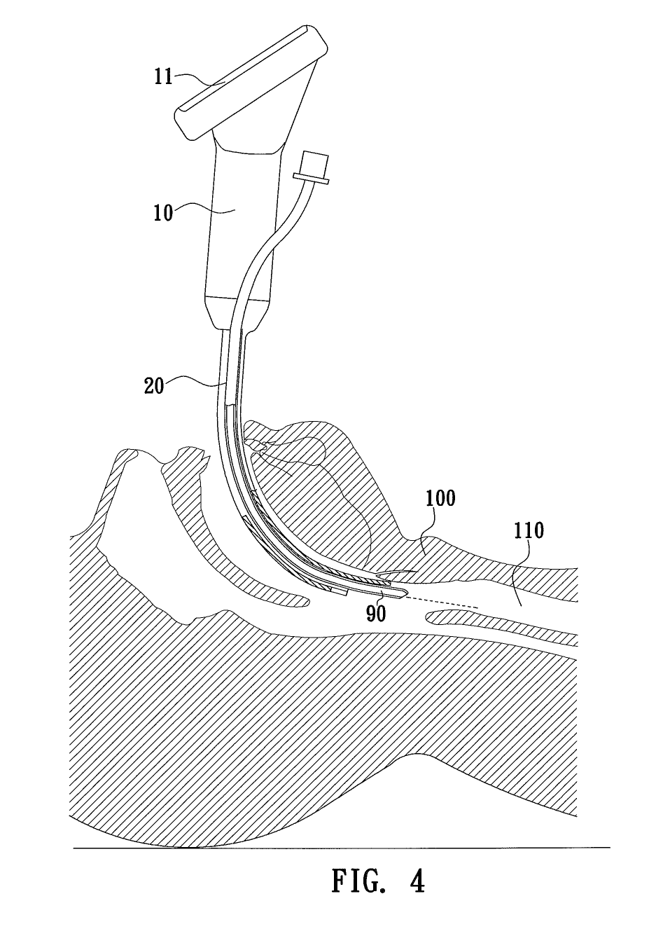

Please refer to FIG. 1 to FIG. 4, related to one embodiment of the tracheal intubation device of the present invention, wherein FIG. 1 is a schematic drawing illustrating the combination of the tracheal intubation device of the present invention and the endotracheal tube; FIG. 2 is a schematic drawing of one embodiment of the camera front end of the present invention before being pushed by the endotracheal tube; FIG. 3 is a schematic drawing of one embodiment of the camera front end of the present invention after being pushed by the endotracheal tube; and FIG. 4 is a schematic drawing illustrating the usage state of one embodiment of the tracheal intubation device of the present invention.

As shown in FIG. 1 and FIG. 2, the tracheal intubation device 1 of the present invention can be used by medical personnel to place the endotracheal tube 90 into the trachea of a patient. The tracheal intubation device 1 of the present invention comprises a handle portion 10, a display unit 11, a guiding portion 20, and a bar-shaped camera 30. The display unit 11 is electrically connected to the bar-shaped camera 30. The bar-shaped camera 30 comprises a camera rear end 32 and a camera front end 31, wherein the camera rear end 32 is electrically connected to the display unit 11 and the image captured by the bar-shaped camera 30 can be displayed via the display unit 11. The camera front end 31 comprises a bended portion 311 and at least one guiding pin 312. The guiding portion 20 comprises a front end 21, a rear end 22, a slot 23, a barrier 24, and an arc-shaped partition 25, wherein the front end 21 is detachably connected to the handle portion 10. The bar-shaped camera 30 and the endotracheal tube 90 can be accommodated and move within the guiding portion 20. It is noted that, as shown in FIG. 1 and FIG. 2, when the endotracheal tube 90 has yet to move within the guiding portion 20, the endotracheal tube 90 is located at the rear of the camera front end 31 to ensure that the endotracheal tube 90 can be placed into the trachea of a patient after the glottis of the trachea of a patient is observed by medical personnel.

According to one embodiment of the present invention, as shown in FIG. 2, the slot 23 is located at the rear end 22 of the guiding portion 20, and both the barrier 24 and arc-shaped partition 25 are located at the bilateral sides of the rear end 22, wherein the barrier 24 contacts the bar-shaped camera 30 and the arc-shaped partition 25 contacts the endotracheal tube 90. The barrier 24 and the arc-shaped partition 25 are used for confining the bar-shaped camera 30 and the endotracheal tube 90 to move within the guiding portion 20. Furthermore, according to one embodiment of the present invention, the camera front end 31 of the tracheal intubation device 1 of the present invention has two guiding pins 312, both of which are located inside the slot 23. The camera front end 31 can slide relative to the guiding portion 20, guided by the guiding pins 312 and the slot 23. It is noted that, although there are two guiding pins 312 in this embodiment, the present invention is not limited to this number; one guiding pin or more than two guiding pins are applicable for the present invention. Moreover, the connection between the guiding portion 20 and the handle portion 10 can be a hook engagement or thread combined with an internal thread.

As shown in FIG. 3, after observing the glottis of the trachea of a patient via the tracheal intubation device 1 of the present invention, medical personnel only have to push the endotracheal tube 90 forward, and the incline at the front end of the endotracheal tube 90 pushes the camera front end 31. Consequently, the camera front end 31 is forced to move away from the moving path of the endotracheal tube 90, and the camera front end 31 moves along the slot 23 to form the state illustrated in FIG. 4. Meanwhile, as shown in FIG. 3, due to the guiding of the slot 23 and the guiding pins 312, the camera front end 31 remains facing toward the glottis of the trachea of the patient to continuously provide images of the location of the glottis of the trachea for medical personnel. Furthermore, after the endotracheal tube 90 is pushed into the trachea 110, the tracheal intubation device 1 of the present invention can be slightly rotated by medical personnel to separate the endotracheal tube 90 from the arc-shaped partition 25. After that, the tracheal intubation device 1 of the present invention can be withdrawn from the mouth of the patient 100. It is noted that, after completion of the intubation, the guiding portion 20 can be separated from the handle portion 10 and discarded or sterilized to ensure medical hygiene.

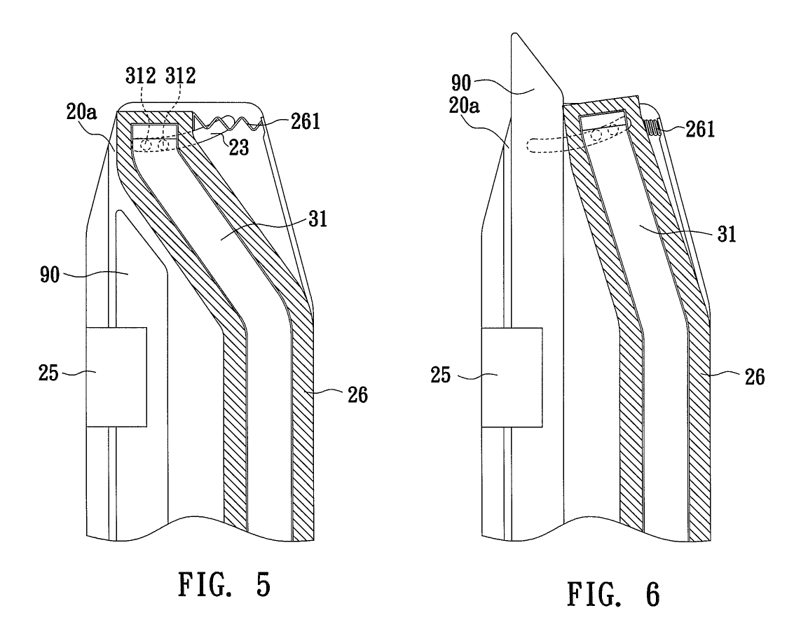

Please refer to FIG. 5 and FIG. 6, wherein FIG. 5 is a schematic drawing of another embodiment of the camera front end before being pushed by the endotracheal tube; FIG. is a schematic drawing of another embodiment of the camera front end after being pushed by the endotracheal tube.

As shown in FIG. 5, the guiding portion 20a in this embodiment of the present invention further comprises a sealing cover 26 for covering the bar-shaped camera 30, and the sealing cover 26 comprises a stretchable portion 261. When the sealing cover 26 has not been pushed by the endotracheal tube 90, the stretchable portion 261 is in a natural elongation state, and the endotracheal tube 90 is still situated at the rear of the camera front end 31. It is noted that the sealing cover 26 of this embodiment is made of transparent plastic, and the stretchable portion 261 is a device that is capable of bearing external forces. The operating pattern of the stretchable portion 261 is similar to that of the bellows of an accordion.

As shown in FIG. 6, after observing the glottis of the trachea of the patient via the tracheal intubation device 1 of the present invention, medical personnel can push the endotracheal tube 90 forward, and the incline of the front end of the endotracheal tube 90 consequently pushes the sealing cover 26 such that the camera front end 31 moves along the slot 23. Meanwhile, the stretchable portion 261 is pressed by pushing force from the front end of the endotracheal tube 90 to force the sealing cover 26 away from the moving path of the endotracheal tube 90. Due to the guiding from the slot 23 and the guiding pins 312, the camera front end 31 continues to face the glottis of the trachea of the patient, as shown in FIG. 6, to continuously provide images of the location of the glottis of the trachea for medical personnel. Furthermore, after the endotracheal tube 90 is pushed into the trachea 110, the tracheal intubation device 1 of the present invention can be slightly rotated by medical personnel to separate the endotracheal tube 90 from the arc-shaped partition 25. After that, the tracheal intubation device 1 of the present invention can be withdrawn from the mouth of the patient 100. It is noted that, after intubation, the guiding portion 20 can be detached and sterilized or discarded to ensure medical hygiene.

It must be noted that the above-mentioned embodiments are only for illustration. It is intended that the present invention covers modifications and variations of this invention provided that they fall within the scope of the following claims and their equivalents. Therefore, it will be apparent to those skilled in the art that various modifications can be made to the structure of the present invention without departing from the scope or spirit of the invention.

What is claimed is:

1. A tracheal intubation device for placing an endotracheal tube into the trachea of a patient, the tracheal intubation device comprising:
a handle portion;
a display unit;
a bar-shaped camera for capturing an image electrically connected to the display unit, the bar-shaped camera comprising:
a camera rear end electrically connected to the display unit, and the image captured by the bar-shaped camera can be displayed via the display unit; and
a camera front end comprising a bended portion; and
a guiding portion comprising a front end and a rear end, wherein the front end is detachably connected to the handle portion, and the bar-shaped camera and the endotracheal tube can be accommodated and move within the guiding portion.

2. The tracheal intubation device as claimed in claim 1, wherein the camera front end further comprises at least one guiding pin.

3. The tracheal intubation device as claimed in claim 2, wherein the guiding portion further comprises a slot located at the rear end for accommodating the at least one guiding pin for allowing the camera front end to slide relative to the guiding portion.

4. The tracheal intubation device as claimed in claim 3, wherein when the camera front end is pushed by the endotracheal tube, the camera front end moves along the slot.

5. The tracheal intubation device as claimed in claim 4, wherein the guiding portion further comprises a barrier and an arc-shaped partition, wherein the barrier and the arc-shaped partition are located at the bilateral sides of the front end, the barrier contacts the bar-shaped camera, and the arc-shaped partition contacts the endotracheal tube.

6. The tracheal intubation device as claimed in claim 3, wherein the guiding portion further comprises a sealing cover for covering the bar-shaped camera.

7. The tracheal intubation device as claimed in claim 6, wherein when the sealing cover is pushed by the endotracheal tube, the camera front end moves along the slot.

8. The tracheal intubation device as claimed in claim 7, wherein the sealing cover comprises a stretchable portion; when the sealing cover is pushed by the endotracheal tube, the stretchable portion is pressed.

9. The tracheal intubation device as claimed in claim 8, wherein the guiding portion comprises an arc-shaped partition for contacting the endotracheal tube.
